(19) United States
(12) Patent Application Publication
(10) Pub. No.: US 2022/0378983 A1
(43) Pub. Date: Dec. 1, 2022

(54) DERMAL FILLER COMPOSITION

(71) Applicant: BIOMED AESTHETICS B.V., Nijmegen (NL)

(72) Inventors: Jasper Johannes Franciscus VAN GOOL, Grave (NL); Julius Marcus VAN DIJCK, Nijmegen (NL); Martinez Marcela Armine GARCIA, Nijmegen (NL); Antonius Andreas Maria VOERMANS, Nijmegen (NL)

(21) Appl. No.: 17/769,699

(22) PCT Filed: Oct. 19, 2020
(86) PCT No.: PCT/NL2020/050640

§ 371 Date: Apr. 15, 2022

(30) Foreign Application Priority Data
Oct. 18, 2019 (NL) 2024060

(51) Int. Cl. A61L 27/48; A61L 27/52; A61L 27/54; A61Q 19/08

(52) U.S. Cl. CPC A61L 27/48; A61L 27/52; A61L 27/54; A61Q 19/08

(57) ABSTRACT

The invention relates to a dermal filler composition in the form of a gel, comprising a carrier fluid comprising water and/or a polyalcohol; cross-linked hyaluronic acid; and spherical microparticles of cross-linked hyaluronic acid having an average diameter in the range of 10-200 μm. The filler provides a volumizing effect as well as a biostimulating effect when injected into skin tissue.

### FIELD OF THE INVENTION

The invention relates to a dermal filler composition, to a method of preparing such composition, to a dermal filler composition for use in the treatment of wrinkles and to a dermal filler composition for use in medical therapy.

### BACKGROUND

Treatment of wrinkles and other lines of the skin often occurs by injecting a dermal filler composition in skin tissue. Such compositions may act either as a volumizer that simply fills a wrinkle, or as a biostimulator that actively induces the formation of collagen once injected. The effect of a volumizer is immediate but does not last long (typically less than one year). On the other hand, the effects of biostimulation manifest only after months and last longer than those of a volumizer.

Unfortunately, the injection of a dermal filler may give complications. The most common side effects are local injection related side effects which manifest as edema, pain, erythema, itching and ecchymosis. These adverse side effects are mild and usually last less than one week but are nevertheless uncomfortable. More severe complications may also occur, but are rare. For example, vascular occlusion may occur within hours or days, leading to local tissue necrosis or embolization of blood vessels. On the longer term, dyspigmentation and scarring may manifest as an adverse side-effect of repeated dermal filler injections.

Ongoing efforts to diminish the occurrence of complications such as pain, irritation and inflammation are thwarted by the desire to use biostimulators, because the underlying mechanism of many biostimulators is that tissue is activated or even inflamed by the injected biostimulator. Therefore, there is a need for dermal fillers in which biostimulation does not go hand in hand with irritation and inflammation. Moreover, many biostimulators are poorly biodegradable, which makes them difficult to remove when undesired side effects occur at the site of injection.

Further, it has been proven difficult to combine a volumizer and a biostimulator in one dermal filler composition. The advantage of such combination would be that its effect is more constant in time, because when the volumizing effect is ending, the biostimulating effect takes over.

### SUMMARY OF THE INVENTION

It is therefore an objective of the present invention to provide a dermal filler composition that causes less pain, irritation and inflammation upon injection in the skin. It is in particular an objective that the composition has an improved biodegradability compared to known dermal fillers. It is also an objective to combine a volumizer and a biostimulator in one dermal filler composition.

It has now been found that one or more of these objectives can be reached by applying a particular dermal filler composition.

Accordingly, the present invention relates to a dermal filler composition in the form of a gel, comprising
a carrier fluid comprising water and/or a polyalcohol;
a gel component of cross-linked hyaluronic acid;
spherical microparticles of cross-linked hyaluronic acid having an average diameter in the range of 10-200 μm.

### BRIEF DESCRIPTION OF THE DRAWINGS

FIG. 1 displays a micrograph of the microparticles that are used in a dermal filler composition according to the invention.

FIG. 2 displays hematoxylin and eosin stained tissues obtained at several intervals after the injection of different hyaluronic-acid based formulations.

FIG. 3 displays the measured collagen density in tissues at twelve months after the injection of different hyaluronic-acid based formulations.

FIG. 4 displays the progress of the biostimulation by the dermal filler composition of the invention over the 12-month period after injection.

FIG. 5 displays the micrographs of the tissues involved in the biostimulation investigations referred to in FIG. 4.

### DETAILED DESCRIPTION OF THE INVENTION

As used herein, the term “dermal filler” broadly refers to a material or composition designed to add volume to areas of soft tissue deficiency. Therefore, as an equivalent term, the term “soft tissue filler” may also be used. Within the meaning of the present invention, the term “soft tissue” generally relates to tissues that connect, support, or surround other structures and organs of the body. In the present invention, soft tissues include, for example, muscles, tendons, vocal cords, lining tissue, fibrous tissues, fat, blood vessels, nerves, and synovial tissues. Further, the term “dermal filler” should not be construed as imposing any limitations as to the location and type of injection. It generally encompasses uses at multiple levels beneath the dermis.

A dermal filler of the invention is in the form of a gel, i.e. it is a gel. The term “gel”, as used herein, generally refers to a material having a fluidity between that of a liquid and a solid at mammalian body temperature (typically 37° C.).

A dermal filler of the invention may comprise other ingredients, in particular active pharmaceutical ingredients. For example, it may comprise a local anesthetic such as lidocaine or vitamins (e.g. vitamin B, C, or E).

The carrier fluid is the medium in which the active compounds (e.g. active in the treatment of wrinkles) are present. The carrier fluid comprises water and/or a polyalcohol. By a polyalcohol is meant an alcohol that contains more than one hydroxyl group, such as a diol or a triol.

The carrier fluid is in principle designed to be a physiologically acceptable carrier fluid. When water is present in substantial amounts (e.g. constituting more than 50 wt. % of the carried fluid), then the carrier fluid is typically buffered at or around physiological pH, e.g. with a physiological saline solution such as phosphate buffered saline (PBS). Other suitable buffers are, for example, Ringer's solution (typically comprising sodium chloride, potassium chloride, calcium chloride and sodium bicarbonate) or Tyrode's solution (typically comprising sodium chloride, potassium chloride, calcium chloride, magnesium chloride, sodium dihydrogen phosphate and sodium bicarbonate).

The pH of such aqueous gel of the invention is usually in the range of 6.4 to 7.8, in particular in the range of 6.8 to 7.4. Such pH may be reached by applying a buffer as provided above having an appropriate pH, or by setting the pH at a desired value by using appropriate amounts of acid and/or base.

The content of carrier fluid in a dermal filler of the invention is usually at least 50 wt. %, based on the total weight of the dermal filler as such. The content may also be at least 60 wt. %, at least 70 wt. %, at least 90 wt. %, at least 95 wt. %, at least 97.5 wt. %, at least 98 wt. %, at least 98.5 wt. %, or at least 99 wt. %. The content may also be 99 wt. % or less, 98 wt. % or less, 95 wt. % or less, 90 wt. % or less, 85 wt. % or less or 75 wt. % or less. Preferably, the content is in the range of 90-98 wt. %.

The polyalcohol in a dermal filler of the invention may be selected from the group of ethylene glycol, glycerol, 1,3 propanediol, 1,4 butanediol, mannitol, sorbitol and poly(ethylene glycol).

The gel properties of a dermal filler of the invention are mostly derived from the gel of cross-linked hyaluronic acid, which gel is a component of the filler. The hyaluronic acid is cross-linked to such extent that it has the properties of a gel. The skilled person knows how to arrive at such gel without exerting inventive efforts and without undue experimentation.

In the gel of cross-linked hyaluronic acid, the mass average molecular mass (M_{w}) of the hyaluronic acid is usually at least 50 kDa. Typically, it is in the range of 100-10,000 kDa. Preferably it is in the range of 200-5,000 kDa or in the range of 500-3,000 kDa.

In a dermal filler composition of the invention, the content of the cross-linked hyaluronic acid that constitutes the gel of cross-linked hyaluronic acid is usually in the range of 0.1-10 wt. % of cross-linked hyaluronic acid, in particular in the range of 0.5-5.0 wt. %, more in particular in the range of 1.0-3.5 wt. %, based on the total weight of the dermal filler as such.

The cross-links in the cross-linked hyaluronic acid (of the gel component) are usually chemical cross-links. These are formed by reaction of the hyaluronic acid with a chemical cross-linking agent. For example, such cross-linking agent is a diglycidyl ether (e.g. 1,2-ethanediol diglycidyl ether or 1,4-butanediol diglycidyl ether) or a di-epoxyalkane (e.g. 1-(2,3-epoxypropyl)-2,3-epoxycyclohexane or 1,2,7,8-diepoxyoctane), Preferably, the cross-linking agent is divinyl sulfone or 1,4-butanediol diglycidyl ether.

The cross-links that result from these cross-linking agents are commonly said to be derived from the respective cross-linking agent. Accordingly, in a dermal filler of the invention, the chemical cross-links of the hyaluronic acid are typically derived from a cross-linking agent selected from the group of diglycidyl ethers and di-epoxyalkanes, preferably from 1,4-butanediol diglycidyl ether or divinyl sulfone.

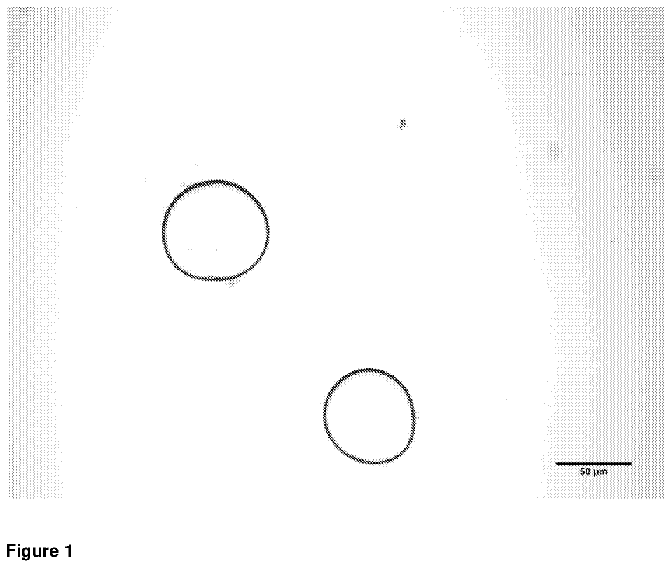

The spherical microparticles in a dermal filler composition of the invention differ from microparticles that are used in known dermal filler compositions in that they have a different shape. Known hyaluronic acid microparticles in such dermal filler applications are non-spherical, while those applied in the invention are spherical, as demonstrated in FIG. 1. Moreover, the microparticles of the invention are also rather soft and may easily and reversibly deform when pressed against. They easily swell or shrink when they absorb or release water, respectively. These differences are the result of different processes of manufacturing the microparticles. In the art, this concerns the cross-linking of hyaluronic acid to form a gel, after which the gel is pulverized into the microparticles by e.g. grinding in a mortar (as in e.g. WO2018/159983A1). Microparticles thus obtained have an irregular shape, e.g. they have sharp edges and high aspect ratio's.

Microparticles of the invention are prepared in a fundamentally different way. The hyaluronic acid is dissolved in an aqueous medium. A water/ethyl acetate emulsion is made from this solution, yielding water droplets comprising the dissolved hyaluronic acid. Being slightly soluble in ethyl acetate, the water is drawn out of the droplets by the ethyl acetate, leaving behind globules of hyaluronic acid. Subsequently, the hyaluronic acid in the created globules is cross-linked. Further work-up then yields the spherical microparticles of cross-linked hyaluronic acid; the exposure to excess water in the work-up makes that they can swell substantially. These particles are thus highly regular and quite soft.

In some cases, the microparticles may deviate slightly from perfectly spherical. For example, the ratio of the shortest diameter to the longest diameter is at least 0.75, at least 0.80, at least 0.85, at least 0.90, at least 0.95, at least 0.97, at least 0.98, at least 0.99 or at least 0.995.

In animal studies (see Example 6), it was found that the presence of spherical microparticles in the composition resulted in an increased collagen formation as compared to compositions comprising only the gel of cross-linked hyaluronic acid and linear hyaluronic acid (and no microparticles) over a period of up to one year. This is a strong indication that spherical microparticles have a biostimulating effect. It was also observed that the injection of the composition comprising the spherical microparticles gave the same level of inflammation as the injection of a composition that lacks the spherical microparticles. This is surprising, since compositions with the irregular microparticles are known for effecting a high level of inflammation in a period of up to two weeks after injection. Thus, the application of the spherical microparticles in the dermal filler composition provides an increased biostimulation that does not go hand in hand with an increased inflammation.

In the method of preparation as outlined above, it appeared that the higher the mass average molecular mass of the initial hyaluronic acid is, the more difficult it is to obtain particles of a certain desired size. For example, when the hyaluronic acid had a mass average molecular mass well above 500 kDa, particles with a size in the range of 15-70 μm were difficult to prepare, which manifested e.g. as low particle yields and low process efficiency. It was initially not regarded as an option to lower the mass average molecular mass of the hyaluronic acid, since this is known to cause undesired inflammation side-effects, as is explained in the following.

Hyaluronic acid is known to have different effects on macrophage expression. The macrophage can undergo a phenotype change depending on the mass average molecular mass (M_{w}) of hyaluronic acid that the macrophage is in contact with. It is known that macrophages display a pro-inflammatory response when in contact with hyaluronic acid with lower mass average molecular mass (M_{w}), such as those as low as 500 kDa, 100 kDa, or 10 kDa. On the other hand, macrophages with a higher mass average molecular mass (M_{w}), typically higher than 500 kDa, are known to give a distinct anti-inflammatory response when in contact with hyaluronic acid.

In the context of the present invention, however, this appeared to be a prejudice. This is because it was surprisingly found that a composition of the invention wherein the microparticles comprise hyaluronic acid with a mass average molecular mass (M_{w}) as low as 10 kDa does not result in a significant pro-inflammatory response.

It was also found that the decrease of the mass average molecular mass (M_{w}) did not cancel any of the other beneficial effects of the invention, in particular its effectivity as a biostimulator.

In conclusion, the decrease of the mass average molecular mass (M_{w}) of hyaluronic acid has opened the way to a more viable preparation of the microparticles, without giving in to the effectivity of the composition and without attracting other undesired effects such as an inflammatory response.

Accordingly, in the spherical microparticles of cross-linked hyaluronic acid, the mass average molecular mass (M_{w}) of the hyaluronic acid is usually at least 1.0 kDa. Typically, it is in the range of 1-5,000 kDa. It may also be in the range of 1.2-3,000 kDa, in the range of 5-2,000 kDa, in the range of 2.4-500 kDa, in the range of 5-100 kDa or in the range of 10-1,000 kDa. Preferably, it is in the range of 5-500 kDa, more preferably in the range of 10-100 kDa.

The spherical microparticles in principle contain water. The water content in the microparticles is usually in the range of 50-99 wt. %, typically it is in the range of 75-95 wt. %. It may for example be 99 wt. % or less, 98 wt. % or less, 97 wt. % or less, 95 wt. % or less, 90 wt. % or less, 85 wt. % or less, 80 wt. % or less, or 75 wt. % or less. It may also be 60 wt. % or more, 75 wt. % or more, 80 wt. % or more, 85 wt. % or more, 90 wt. % or more, 93 wt. % or more or 95 wt. % or more.

The cross-links in the hyaluronic acid of the spherical microparticles are usually chemical cross-links, and are e.g. derived from a cross-linking agent selected from the group of diglycidyl ethers, di-epoxyalkanes and divinyl sulfone, in particular from 1,4-butanediol diglycidyl ether or divinyl sulfone.

The spherical microparticles in a dermal filler of the invention usually have an average diameter of 300 μm or less, 150 μm or less, 50 μm or less or 40 μm or less. It is usually 10 μm or more, 15 μm or more, 20 μm or more, 25 μm or more, 30 μm or more, 40 μm or more or 50 μm or more. Typically, it is in the range of 15-100 μm, in particular in the range of 15-70 μm, more in particular in the range of 20-55 μm, and even more in particular in the range of 30-50 μm.

The content of spherical microparticles in a dermal filler of the invention is usually in the range of 0.4-10 vol. %, in particular in the range of 0.5-8 vol. %, more in particular in the range of 1-6 vol. %.

A dermal filler of the invention may have undergone a sterilization process to provide the dermal filler as a sterile dermal filler. For example, it is sterilized by exposure to high temperature (e.g. by steam sterilization) or to high energy radiation (e.g. gamma irradiation).

The viscosity of a dermal filler of the invention may be tuned by varying certain characteristics of the dermal filler, such as the degree of cross-linking of the hyaluronic acid used in the gel component, the amount and size of the microparticles, and the relative abundancy of the different components, in particular of 1) the gel of cross-linked hyaluronic acid; 2) the microparticles; and 3) an eventual additive with an influence on the viscosity, such as linear hyaluronic acid (vide infra). Depending on the specific application of the dermal filler, a higher or a lower viscosity can be set.

The dynamic viscosity of a gel of the invention is usually in the range of 10-1100 Pa·s. It may also be in the range of 20-800 Pa·s or in the range of 30-700 Pa·s. A person skilled in the art will be able to find the conditions that are required for reaching a certain viscosity by routine experimentation and without exerting an inventive effort.

The Dynamic Modulus (Storage Modulus) of a gel of the invention is usually in the range of 1-3,000 Pa, in particular in the range of 5-2,500 Pa, more in particular in the range of 15-2,000 Pa and even more in particular in the range of 20-1,500 Pa.

A dermal filler composition of the invention may comprise linear hyaluronic acid. The main purpose of this additive is that it may be used to tune the viscosity and so optimize the injectability of the filler.

When linear hyaluronic acid is present, it usually has a mass average molecular mass (M_{w}) of at least 250 kDa. Typically, it is in the range of 300-10,000 kDa. Preferably it is in the range of 500-5,000 kDa or in the range of 800-4,000 kDa.

When linear hyaluronic acid is present in a dermal filler of the invention, it is usually present in the range of 0.05-5.0 wt. %, in particular in the range of 0.1-2.0 wt. %, based on the total weight of the dermal filler as such.

The linear hyaluronic acid, when present, and the cross-linked hyaluronic acid are usually present in a mass ratio in the range of 1.0:0.25 to 1.0:15.0, in particular in the range of 1.0:1.0 to 1.0:10.0, based on their dry matter content.

It is an advantage of the dermal filler of the invention that the filler provides a volumizing effect as well as a biostimulating effect. After application of the filler, the volumizing effect manifests in a shorter term than the biostimulation. This gives a more constant appearance of a filled wrinkle than when there is only a volumizing effect or only a biostimulating effect.

It is also an advantage that a patient who is injected with a dermal filler of the invention experiences less side-effects than when a conventional dermal filler is injected, in particular less pain and/or less inflammation. It is all the more an advantage that this decrease of side-effects does not go at the expense of the effectivity of biostimulation. In other words, a dermal filler composition of the invention provides an increased biostimulation that does not go hand in hand with an increased inflammation and/or with a continuous inflammation.

A dermal filler composition of the invention contains only hyaluronic acid and derivatives thereof as active substances for the treatment of wrinkles, i.e. substances that contribute to sustained volumizing effects and biostimulating effects in the skin. For example, the presence of other active substances such as a local anesthetic or certain vitamins is not considered to have a sustained volumizing effect, just as the injected carrier fluid. This makes that the composition of the invention is fully biodegradable. Moreover, as demonstrated in the Examples, this is combined with a high safety profile and a reduction of the chances on, and severity of, undesired side-effects.

The invention further relates to a method for preparing a dermal filler composition in the form of a gel, comprising
preparing a gel of cross-linked hyaluronic acid;
preparing spherical microparticles of cross-linked hyaluronic acid, wherein the spherical microparticles have an average diameter in the range of 10-200 μm when they are present in the gel;
mixing the cross-linked hyaluronic acid and the spherical microparticles with a carrier fluid comprising water and/or a polyalcohol to form the gel.

In a method of the invention, the gel component of cross-linked hyaluronic acid and the spherical microparticles are usually prepared separately, after which they are mixed with the carrier fluid to form the gel of the invention. Thus, there are then at least three components that are combined in a method of the invention (e.g. optionally also the linear hyaluronic acid).

There are multiple modes of combining these components. The three polymers may be void of water when combined, but one or more of them may also contain water upon combining the four components.

For example, the cross-linked hyaluronic acid may be contained in the carrier fluid to form gel, while the linear hyaluronic acid and the microparticles may added to this gel as a dry solid. The linear hyaluronic acid is often applied as a dry solid, since it is commonly purchased as a dry powder that is ready for use in the method of the invention. The microparticles are typically prepared in an aqueous environment, which makes it convenient to apply them in wet form in a method of the invention. They may however also be dried prior to combining them with the other components.

The cross-linked hyaluronic acid is usually prepared by treating linear hyaluronic acid with a chemical cross-linking agent, e.g. divinyl sulfone or 1,4-butanediol diglycidyl ether. Analogously, the spherical microparticles of hyaluronic acid are usually also prepared by cross-linking the linear hyaluronic acid. The process for preparing the spherical microparticles is performed in such manner that the microparticles have an average diameter in the range of 10-200 μm when they are present in the final product of the process, which is the dermal filler. Their average diameter, when measured directly after their preparation in an aqueous environment, may be different from the average diameter of the microparticles in the final product, especially when the aqueous environment of their preparation is different from the carrier fluid in the final product (the latter may e.g. comprise a buffer, while the former may lack such buffer).

The spherical microparticles are usually prepared in such manner that their average diameter in the final product is in the range of 15-70 μm, in particular in the range of 20-55 μm, more in particular in the range of 30-50 μm. This means that during the particle preparation, there is already accounted for a change in size of the particles that occurs later on when the particles are used to prepare the composition of the invention, in particular when they are mixed with the cross-linked hyaluronic acid and the carrier fluid. Such change typically occurs when the amount of water that is contained by the microparticle changes, for example due to different concentrations of the components present, including salt concentration and pH. The skilled person knows which particle size should be initially present in order to arrive at the desired particle size in the final dermal filler composition.

The preparation of the microparticles may include the use of a sieve. The spherical microparticles are then sieved over a plurality of sieves to yield particles with an appropriate average diameter. When a sieving step is performed, the spherical microparticles are usually in a wet state, i.e. they comprise water.

A method of the invention typically includes a sterilization step, yielding the dermal filler of the invention as a sterile dermal filler. For example, a dermal filler formed according to a method of the invention may be exposed to an elevated temperature, e.g. to a temperature in the range of 80-140° C., in particular in the range of 100-135° C. The temperature and the period of exposure are then chosen such that any micro-organisms are destroyed to a desired extent, whilst not degrading the dermal filler too much. For example, the dermal filler is exposed during 15-20 minutes (e.g. at a temperature in the range of 115-125° C.), or it is exposed during 2-10 minutes (e.g. at a temperature in the range of 130-140° C.).

Sterilization may also be achieved by exposing the gel to high energy radiation, in particular ionizing radiation such as gamma rays, electron rays, X-rays, and the higher ultraviolet part of the electromagnetic spectrum. The dosage to which a gel may be exposed is e.g. 15, 25 or 50 kGy.

The invention further relates to a dermal filler obtainable by the method as described hereinabove.

A dermal filler of the invention is usually applied in the cosmetic field, in particular in the cosmetic treatment of wrinkles and lines of the skin. It may however also find application in the medical field. Accordingly, the invention further relates to a dermal filler as described hereinabove, for use in medical therapy, for use as a medicament and/or for use in medicine.

The invention further relates to a dermal filler as described hereinabove, for use in the treatment of atrophic acne scars, lipodystrophy, stress urinary incontinence, vesicoureteral reflux, vocal fold insufficiency, and/or vocal fold medialization.

The invention further relates to a method for filling of a tissue or increasing the volume of a tissue for cosmetic or therapeutic purposes, comprising administering to a human or animal an effective amount of a dermal filler composition as described hereinabove. The soft tissue may be skin, muscles, tendons, vocal cords, fibrous tissues, fat, blood vessels, nerves, and synovial tissues. The administration is typically performed by injecting the dermal filler with a syringe via a needle into the tissue.

The invention further relates to the use of a dermal filler as described hereinabove for treating a tissue in an individual in need thereof.

The invention further relates to the use of a dermal filler as described hereinabove for the manufacture of a medicament for treating acne scars, lipodystrophy, stress urinary incontinence, vesicoureteral reflux, vocal fold insufficiency, and/or vocal fold medialization in an individual in need thereof.

### EXAMPLES

### Example 1. Preparation of Hyaluronic Acid Gel

Hyaluronic acid gels were prepared containing 1.5 g of 2,600 kDa hyaluronic acid (HA) in 13.5 g of 0.25 M sodium hydroxide (NaOH). After all HA was dissolved, 165 mg of 1,4-butanediol diglycidyl ether (BDDE) was added to the solution and mixed for 5 minutes with a spatula. The solution was put in a plastic cup which was closed off and transferred to an oven at 50° C. for 2 hours. The gel was then placed in an excess amount of PBS and left to hydrate till it reached a HA percentage of 1.5 wt. %.

### Example 2. Preparation of Cross-Linked Microparticles

50 mg of 10 kDa hyaluronic acid was dissolved in 2 mL 0.005 NaOH together with 7.5 mg of divinyl sulfone and left at room temperature for 2 hours. Then, 400 mL of ethyl acetate in an 800 mL beaker was stirred at 2,000 rpm with an overhead stirrer and the hyaluronic acid solution was added through a 30 G needle over a course of 2 minutes. The solution was left to stir for 1 hour. Afterwards the solution was left at room temperature for 24 hours. After purification (removal of ethyl acetate in vacuo) the particles were filtrated and washed. During the washing the particles swelled substantially, yielding soft hyaluronic acid microparticles.

### Example 3. Combining all Three Components

22 g of the hydrated gel prepared in example 1 was mixed with 50 mg of 1,600 kDa hyaluronic acid powder for 5 minutes with a spatula. Afterwards, 90 mg of the particles prepared in example 2 were added and the resulting mixture was stirred for 5 minutes.

### Example 4. Microparticles Size Analysis

The microparticles prepared in example 2 were analyzed using a Leica upright DM2500 light microscope. With a bright field 200× magnification the particles were examined. The morphology as well as the size was analyzed using Image J. An average spherical particle size of 30-50 μm was obtained. FIG. 1 displays a micrograph of the obtained microparticles.

### Example 5. Rheometric Analysis of the Gel

The product made in example 3 was analyzed with a Discovery Hybrid Rheometer (TA Instruments). With a 1,200 μm gap height at 25° C., the storage and loss modulus were measured. The plate had a 25 mm diameter and a 1% strain was applied. A frequency sweep was performed from 0.1 Hz to 5.0 Hz. At 5.0 Hz the gel had a storage modulus of 372 Pa and a loss modulus of 52 Pa.

### Example 6. Biological Response—Animal Study

### 6.1. Set-Up

An animal study was conducted to test the safety and effectiveness of the dermal filler composition of the invention. The in vivo study was conducted from Jun. 2, 2019 to Jun. 2, 2020 at Hangzhou Huibo Science and Technology Co., Ltd in Hangzhou China. The composition was tested on New Zealand white rabbits and progress of the study was monitored at three different timepoints: 3, 6, and 12 months. Three different gel formulations (A, B and C) were tested:
A) cross-linked hyaluronic acid gel with linear hyaluronic acid;
B) hyaluronic acid microparticles with linear hyaluronic acid; and
C) cross-linked hyaluronic acid gel with linear hyaluronic acid and hyaluronic acid microparticles (i.e. the composition of the invention).

The three formulations were prepared according to the procedures described in Examples 1-3 above (formulation B was prepared according to the procedure of Example 3, with the exception that cross-linked hyaluronic acid was left out of the procedure).

The primary aim of testing these formulations was to investigate the effect of the microparticles on the biostimulating effect of the dermal filler composition. At the same time, any side effects such as inflammation, rejection and encapsulation were monitored.

A total of 21 New Zealand white rabbits were used in this study. The rabbits were allocated as follows. A total of one rabbit per timepoint was used for formulation A; three rabbits per timepoint were used for formulations B and C. A volume of 0.1 to 0.2 mL was infiltrated in the dorsum of each rabbit, approximately 5 cm away from the spinal column, between the front and back extremities of the animal.

The two staining used to verify the safety and effectiveness of the hyaluronic acid gel were hematoxylin and eosin (H&E) along with Pricosirius red (PSR), respectively. The H&E staining provided a very detailed status of the tissue structure after the infiltration of the gel, and the PSR staining highlighted the presence of collagen fibers at each specific region of interest.

All images were processed using the software Image J. All images were analyzed at the same scale and magnification of 10×. The images were processed through Colour deconvolution, using H&E staining within the scroll down option. The region of interest (ROI) and thresholding parameters were fixed and used across all pictures. The quantity data collected was then processed in Excel and plotted as seen in the Figures.

In the following, the term ‘hyaluronic acid’ is sometimes abbreviated as ‘HA’; the term ‘microparticles’ is sometimes abbreviated as MP.

### 6.2. Results

First, the obtained data were analyzed on the volumizing effectivity of the dermal filler composition. This is the increase of volume caused by the injection of cross-linked hyaluronic acid. A volumizing effect is generally known to be the highest directly after injection. Thereafter, cross-linked hyaluronic acid degrades, which manifests as a decrease of the volumizing. The data were also analyzed on the occurrence of side-effects occurred such as inflammation, rejection, encapsulation.

Formulation A or formulation C was injected and the skin was monitored over time. Tissues of the rabbits were analyzed at certain time intervals (3, 6 and 12 months) after the injection.

In the first weeks after the injection, any irritation/inflammation that has been observed was equally divided over the tissues that were treated with each formulation.

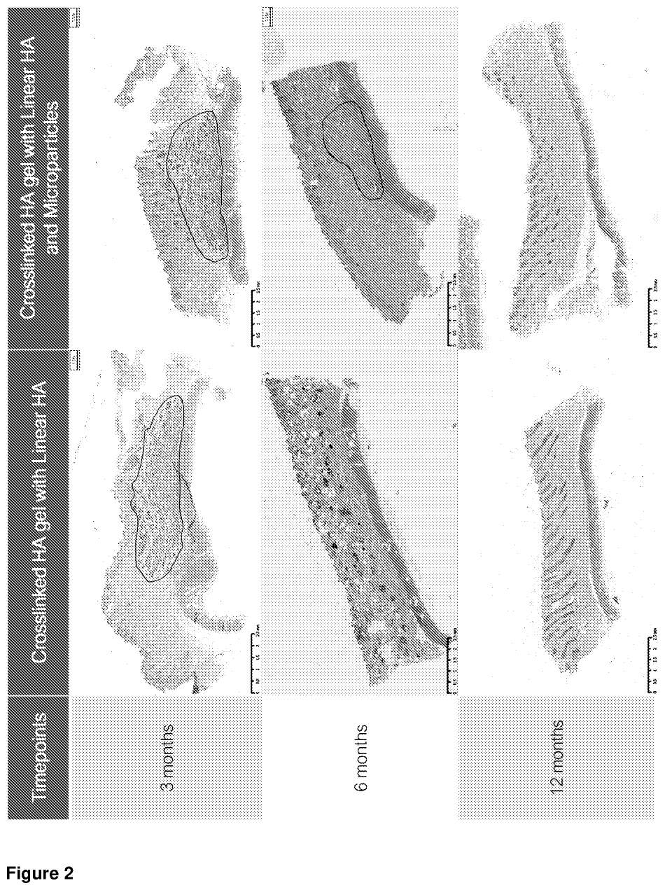

FIG. 2 displays H&E stained dermal layer tissues obtained at 3, 6 and 12 months after the injection of formulation A or formulation C (the scale bars all run from 0-2.5 mm over five sections of 0.5 mm). The gel is identified as a darker area, of which the contours are indicated with a black line. After three months, much of the gel is still present. No rejection from the skin has occurred at the injection site, nor at surrounding areas. The dermal layer has fully embraced the surface area of the gel, not only from the perimeter but also within/across it. The pictures after six months show a fainter appearance of the gel. After one year, no gel was left at the injection site, indicating complete degradation of the gel. Such degradation profile is in line with that of conventional volumizing compositions. Encapsulation of the gel through fibrotic tissue was not observed at any time, indicating that no immunologic response has been triggered. In conclusion, these tests indicate that a composition of the invention is an effective volumizer and that the presence of the microparticles herein do not give undesired side-effects upon and after injection. This is all the more surprising in view of the mass average molecular mass (M_{w}) of hyaluronic acid in the particles. The M_{w} is only 10 kDa, a value that is normally regarded as unsuitable since it triggers a pro-inflammatory response in the body. It appears that this low value may be key to the effectivity of the biostimulation as well as to the low inflammatory response of a dermal filler composition of the invention.

Second, the obtained data were analyzed on the biostimulating effectivity of the dermal filler composition. This is the extent to which the injected composition stimulates the formation of collagen. To this end, the amount of collagen fibers within each tissue sample is quantified by measuring the collagen density at the injection site, which is based on PSR polarized images of the tissues.

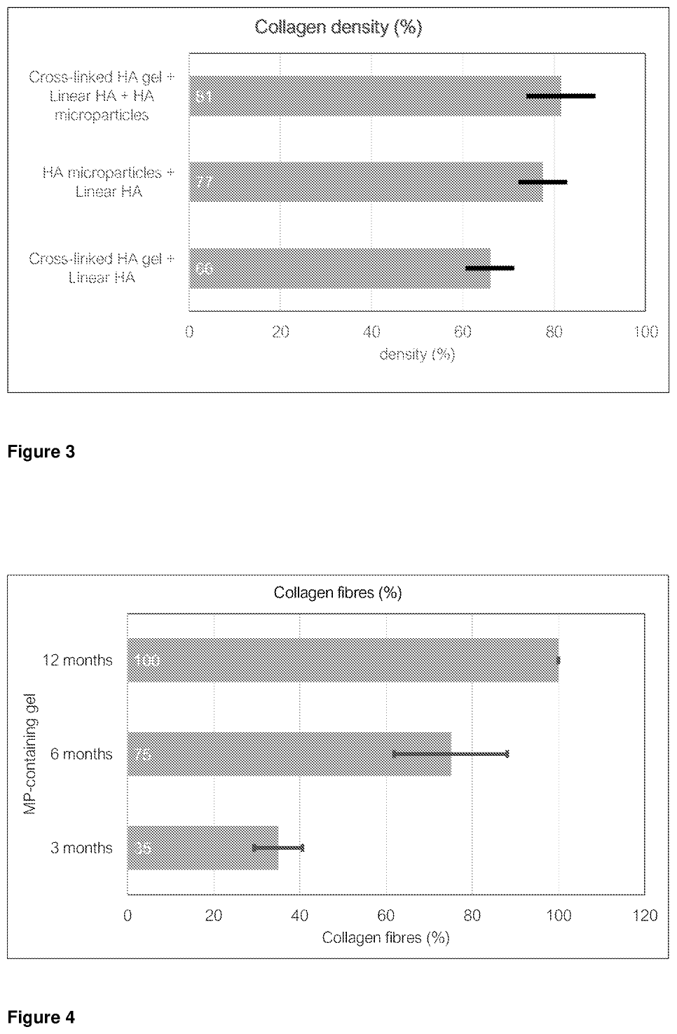

FIG. 3 displays the measured collagen density in tissues at twelve months after the injection with formulation A, formulation B or formulation C. With formulation A providing a reference value corresponding to the absence of biostimulation (lower bar), it follows from FIG. 3 that formulations B and C indeed initiate the formation of collagen (middle bar and upper bar, respectively). This is supported by visual inspection of the tissues themselves, as tissues injected with formulation B or C have a more dense appearance and contain less voids than tissues injected with formulation A.

Moreover, formulation C (of the invention) appears to perform even better than formulation B as regards the biostimulation. This is an indication of a synergistic effect of the microparticles and the cross-linked hyaluronic acid gel. Possibly, the gel inhibits the bio-degradation of the microparticles so that there is a prolonged activity of the microparticles.

Third, the obtained data were analyzed on the progress of the biostimulation by the dermal filler composition of the invention (formulation C) over the 12-month period after injection. It is generally known that it takes some time before biostimulation has a measurable effect, e.g. one or more months.

FIG. 4 demonstrates such time profile for formulation C, as the effect is moderate after three months and has almost tripled in the nine months thereafter.

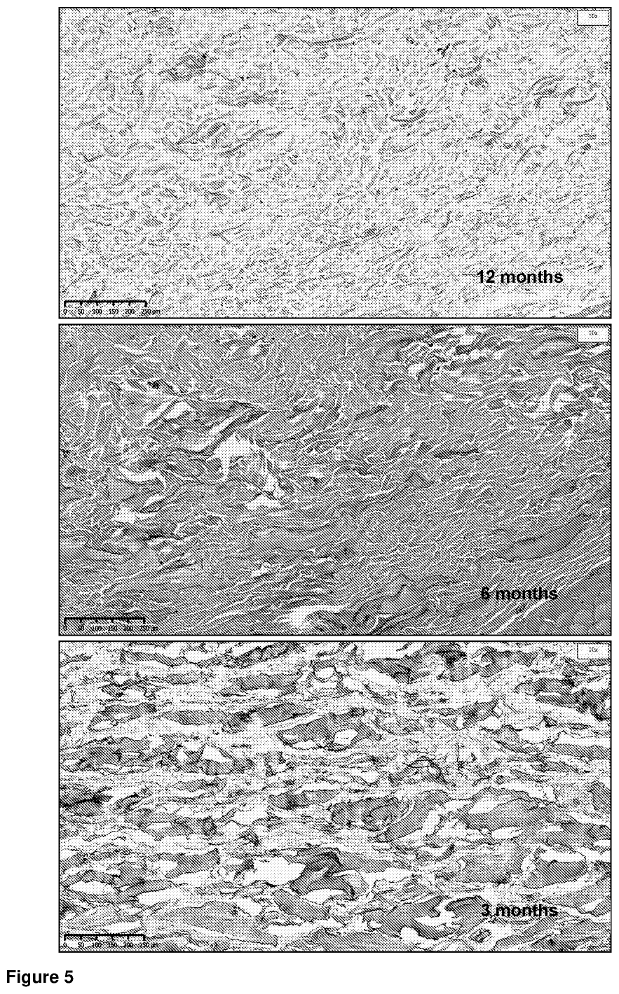

FIG. 5 displays the micrographs of the tissues that correspond to the bars of FIG. 4, revealing more homogeneity and less voids as time progresses (the scale bars all run from 0-250 μm over five sections of 50 μm). This also evidences the biostimulating effect of the dermal filler composition of the invention.

### 6.3. Conclusions

The above data yield the following conclusions.
1. The composition of the invention has an initial volumizing effect which is in the course of 12 months taken over by a biostimulating effect. The combination of both effects is that the filling is perceived constant over time.
2. The microparticles are responsible for the biostimulating effect and act herein synergistically with the cross-linked hyaluronic acid gel.
3. The composition of the invention exhibits the properties under 1) and 2) without giving undesired side-effects upon and after injection. This is in contrast to the known compositions that comprise the irregularly-shaped hyaluronic particles. It is contemplated that the smoothness of the spherical microparticles in the filler of the invention is responsible for the absence of the side-effects.

1. Dermal filler composition in the form of a gel, comprising
a carrier fluid comprising water and/or a polyalcohol;
a gel of cross-linked hyaluronic acid;
spherical microparticles of cross-linked hyaluronic acid having an average diameter in the range of 10-200 pm.

2. Dermal filler composition according to claim 1, wherein the content of the carrier fluid is at least 90 wt. %, based on the weight of the dermal filler as such, preferably at least 95 wt. %.

3. Dermal filler composition according to claim 1, wherein the content of the cross-linked hyaluronic acid that constitutes the gel of cross-linked hyaluronic acid is in the range of 0.1-10 wt. %, in particular in the range of 0.5-5.0 wt. %, more in particular in the range of 1.0-3.5 wt. %, based on the total weight of the dermal filler.

4. Dermal filler composition according to claim 1, wherein, in the gel of cross-linked hyaluronic acid, the mass average molecular mass (Mw) of the hyaluronic acid is in the range of 200-5.000 kDa, in particular in the range of 500-3,000 kDa.

5. Dermal filler composition according to claim 1, wherein, in the spherical microparticles of cross-linked hyaluronic acid, the mass average molecular mass (M_{w}) of the hyaluronic acid is in the range of 5-500 kDa, in particular in the range of 10-100 kDa.

6. Dermal filler composition according to claim 1, wherein the hyaluronic acid in the gel and/or in the microparticles comprises cross-links derived from a cross-linking agent selected from the group of diglycidyl ethers, di-epoxyalkanes and divinyl sulfone, in particular from 1,4-butanediol diglycidyl ether or divinyl sulfone.

7. Dermal filler composition according to claim 1, wherein the spherical microparticles have an average diameter in the range of 15-70 pm, in particular in the range of 20-55 pm, more in particular in the range of 30-50 pm.

8. Dermal filler composition according to claim 1, wherein the content of spherical microparticles in a dermal filler of the invention is in the range of 0.4-10 vol. %, in particular in the range of 0.5-8 vol. %, more in particular in the range of 1-6 vol. %.

9. Dermal filler composition according to claim 1, further comprising linear hyaluronic acid.

10. Dermal filler composition according to claim 9, wherein the mass average molecular mass (M_{w}) of the linear hyaluronic acid is in the range of 500-5,000 kDa, in particular in the range of 800-4,000 kDa.

11. Method for preparing a dermal filler composition in the form of a gel, comprising
preparing a gel of cross-linked hyaluronic acid;
preparing spherical microparticles of cross-linked hyaluronic acid, wherein the microparticles have an average diameter in the range of 10-200 pm when they are present in the gel;
mixing the cross-linked hyaluronic acid and the spherical microparticles with a carrier fluid comprising water and/or a polyalcohol to form the gel.

12. Method according to claim 11, wherein the cross-links are derived from a cross-linking agent selected from the group of diglycidyl ethers, di epoxyalkanes and divinyl sulfone, in particular 1,4-butanediol diglycidyl ether or divinyl sulfone.

13. Method according to claim 11, wherein the spherical microparticles are sieved over a plurality of sieves to yield particles with an appropriate average diameter.

14. Dermal filler composition according to claim 1, for use in medical therapy, cosmetic therapy or aesthetic therapy.

15. Dermal filler composition according to claim 1, for use in the treatment of atrophic acne scars, lipodystrophy, stress urinary incontinence, vesicoureteral reflux, vocal fold insufficiency, and/or vocal fold medialization.

16. Method for filling of a tissue or increasing the volume of a tissue for cosmetic or therapeutic purposes, comprising administering to a human an effective amount of a dermal filler composition according to claim 1.

17. Use of a dermal filler composition according to claim 1 for treating a tissue of an individual.
